(19) United States
(12) Patent Application Publication
(10) Pub. No.: US 2024/0307003 A1
(43) Pub. Date: Sep. 19, 2024

(54) METHOD FOR GENERATING TRAINING DATA FOR USE IN MONITORING A HEALTH PARAMETER OF A PERSON

(71) Applicant: KNOW LABS, INC., Seattle, WA (US)

(72) Inventor: JOHN CRONIN, Seattle, WA (US)

(21) Appl. No.: 18/601,500

(22) Filed: Mar. 11, 2024

Related U.S. Application Data
(60) Provisional application No. 63/490,883, filed on Mar. 17, 2023.

(51) Int. Cl. A61B 5/00; A61B 5/05; A61B 5/145; G16H 40/67

(52) U.S. Cl. CPC A61B 5/7267; A61B 5/05; A61B 5/14532; A61B 5/7225; G16H 40/67

(57) ABSTRACT

A method for generating training data for use in monitoring a health parameter of a person in which a waveform database of glucose waveforms and health parameters is created. The device receives a pulse wave signal that is generated from radio frequency scanning data that corresponds to radio waves that have responded from below the skin surface of a person, wherein the radio frequency scanning data is collected through a two-dimensional array of receive antennas over a range of radio frequencies. Then at least one of the pulse wave signals is extracted in response to using convolution matching to the waveform database to generate an extracted signal. Then the data is labeled corresponding to the extracted signal with a corresponding blood glucose level to generate training data.

### FIELD OF THE DISCLOSURE

The present disclosure is generally related to generating training data for use in monitoring a health parameter of a person.

### BACKGROUND

Diabetes is a medical condition in which a person's blood glucose level, also known as blood sugar level, is persistently elevated. Diabetes can result in severe medical complications, including cardiovascular disease, kidney disease, stroke, foot ulcers, and eye damage if left untreated. Typically, diabetes is caused by either insufficient insulin production by the pancreas, referred to as “Type 1 diabetes,” or improper insulin response by the body's cells referred to as “Type 2 diabetes.” Further, monitoring a person's blood glucose level and administering insulin when a person's blood glucose level is too high to reach the desired level may be part of managing diabetes. Depending on many factors, such as the severity of diabetes and the individual's medical history, a person may need to measure their blood glucose level up to ten times per day. Each year, billions of dollars are spent on equipment and supplies for monitoring blood glucose levels.

Moreover, regular glucose monitoring is a crucial component of diabetes care. Further, measuring blood glucose is generally an invasive procedure by giving a blood sample at a clinic or hospital. Home glucose monitoring is also possible using a variety of devices. The blood sample is obtained by pricking the skin using a tiny instrument. A glucose meter or glucometer is a tiny instrument that measures the sugar in the blood sample. The majority of glucose monitoring methods and devices require a blood sample. Currently, available glucose monitoring devices also require a blood sample, usually by pricking a needle under the skin and then using a glucose meter to determine the glucose level of a patient. These monitoring devices are almost 95 percent accurate. However, such monitoring devices are often prone to contamination. Currently, supervised machine learning data can be used to create and fine tune algorithms that can be used by devices that are less intrusive than current methods of checking glucose levels. Also, current methods of creating the training data for supervised machine learning algorithms use filtering to generate the training data. In order to improve the training data generated, and thus the algorithms, it would be beneficial to use matching to improve the quality of data generated.

### SUMMARY

Systems and methods of generating training data for use in monitoring a health parameter of a person. For example, a method for generating training data for use in monitoring a health parameter of a person in which a waveform database of glucose waveforms and health parameters is created. A pulse wave signal is received that is generated from radio frequency scanning data that corresponds to radio waves that have responded from below the skin surface of a person, wherein the radio frequency scanning data is collected through a two-dimensional array of receive antennas over a range of radio frequencies. Then at least one of the pulse wave signals is extracted in response to using convolution matching to the waveform database to generate an extracted signal. Then the data is labeled corresponding to the extracted signal with a corresponding blood glucose level to generate training data.

In one example, a health monitoring system includes a monitoring device that includes one or more transmit antennas configured to transmit radio-frequency (RF) glucose detection signals into a user and one or more receive antennas configured to detect RF glucose signals that result from the RF glucose detection signals transmitted into the user. An analog-to-digital converter is connected to the one or more receive antennas and receiving the RF glucose signals detected by the one or more receive antennas. A memory stores a standard waveform database with a plurality of standard waveforms. A matching module is stored in the memory that is configured to perform convolution matching. A labeling module is stored in the memory and accesses labels stored in the memory. A label can be assigned to each convolution match stored in the memory.

In another example, a health monitoring method includes generating a waveform database of glucose waveforms and associated health parameter labels; transmitting radio-frequency (RF) glucose detection signals into a user from one or more transmit antennas and detecting, using one or more receive antennas, RF glucose signals that result from the RF glucose detection signals transmitted into the user; converting the detected RF glucose signals from analog signals to digital signals using an analog-to-digital converter connected to the one or more receive antennas; extracting one of the glucose waveforms from the waveform database and performing convolution matching on the extracted glucose waveform and one of the digital signals; storing a convolution match in memory; and labeling data corresponding to the convolution match with a corresponding blood glucose level.

In another example, a method of generating training data for use in monitoring a health parameter of a person includes generating a waveform database of glucose waveforms and health parameter labels; receiving a pulse wave signal that is generated from radio frequency scanning data that corresponds to radio waves that have responded from below the skin surface of a person, wherein the radio frequency scanning data is collected through a two-dimensional array of receive antennas over a range of radio frequencies; storing the pulse wave signal in the waveform database; extracting the pulse wave signal from the waveform database and performing convolution matching to generate an extracted signal; and labeling data corresponding to the extracted signal with a corresponding blood glucose level to generate training data.

### DRAWINGS

FIG. 1: Illustrates a schematic of a system for generating training data for use in monitoring a health parameter of a person, according to an embodiment.

FIG. 2: Illustrates a Device Base Module, according to an embodiment.

FIG. 3: Illustrates a Matching Module, according to an embodiment.

FIG. 4: Illustrates a Labeling Module, according to an embodiment.

### DETAILED DESCRIPTION

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

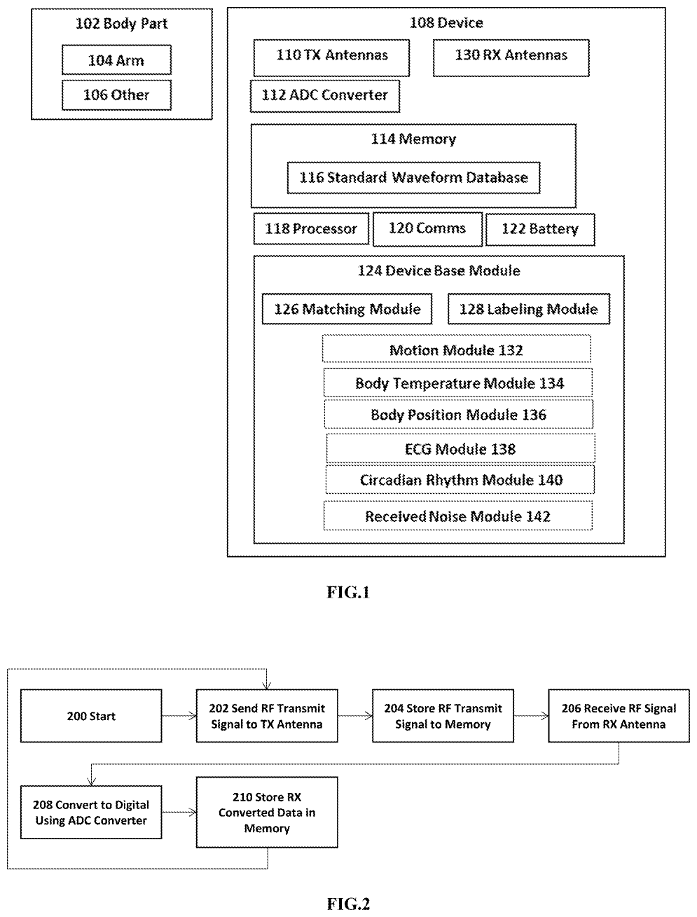

FIG. 1 illustrates a system for generating training data for use in monitoring a health parameter of a person. This system comprises a user with a body part 102 such as any part of a human including a limb or extremity. Further, embodiments may include the user having an arm 104 such as each of the two upper limbs of the human body from the shoulder to the hand. Further, embodiments may include other 106 parts of the body such as a limb or extremity, such as a leg or torso. Further, device 108 may be communicatively coupled to a device network (not shown). The device network may be a software module in a server in the cloud that communicates with the device 108 via a communication module 120. In one embodiment, many or all the functions of device base module 124 may be performed on the device network. In yet another embodiment some of the operations of the device base module 124 would be executed in the device base module 124 and some may be executed in the device network. In yet another embodiment all of the operations of the device base module 124 would be executed in parallel with the device base module 124 (for redundancy) and some may be executed in the device network.

In one embodiment, the device network may be a wireless and/or wired communication channel. The device 108 may be worn by the user. Device 108 may determine health parameters using radio frequency signals in the RF Activated Range. RF Activated Range means frequencies between 500 MHZ and 300 GHZ. In one embodiment, the health parameters may include blood sugar or blood glucose levels. The system may target specific blood vessels using the RF Activated Range radio frequency signals, which generates output signals, and the output signals may correspond to the blood glucose level in the user. In one embodiment, the system may include integrated circuit (IC) devices (not shown) with transmit and/or receive antennas. Monitoring the blood glucose level in blood in the specific blood vessels of the user using the RF Activated Range radio frequency signals involves the transmission of suitable RF Activated Range radio frequency signals below the user's skin surface. Corresponding to the transmission, a responded portion of the RF Activated Range radio frequency signals is received on multiple receive antennas. A responded portion means a received RF signal by an RX antenna occurs within a certain time window of the specific transmitted signal by a TX antenna. The time window may be for instance, 10 ns. Further, the system isolates and/or processes a signal from a particular location of the blood vessels in response to the received RF Activated Range radio frequency signals. The system may output a signal from the received RF Activated Range radio frequency signals that correspond to the blood glucose level in the user. It can be noted that the device 108 may be worn by the user at various locations such as wrist, arm, leg, etc. In one embodiment, the system for monitoring the blood glucose level of the user using the RF Activated Range radio frequency signals involves transmitting RF Activated Range radio frequency signals below the skin surface, receiving a responded portion of the RF Activated Range radio frequency signals on multiple receive antennas, isolating a signal from the RF Activated Range radio frequency signals at a particular location in response to the received RF Activated Range radio frequency signals, and outputting a signal that corresponds to the blood glucose level in the user in response to the isolated signal. In one embodiment, beamforming is used in the receiving process to isolate the RF Activated Range radio frequency signals responded from a specific location on a specific blood vessel to provide a high-quality signal corresponding to the blood glucose levels in the specific blood vessel. In another embodiment, Doppler effect processing may be used to isolate the RF Activated Range radio frequency signals responded from the specific blood vessel's specific location to provide the high-quality signal corresponding to the blood glucose levels in the blood in the specific blood vessel. Also, it should be noted that a correlation algorithm or machine leaning algorithm can be used between the received signal from the RX antenna and a waveform database and or ground truth data. It should be noted that any hybrids of beamforming, doppler or correlation and machine learning could be used.

It can be noted that analog and/or digital signal processing techniques may be used to implement beamforming and/or Doppler effect processing or correlation or machine learning and digital signal processing of the received signals to dynamically adjust a transmitted beam onto the desired location. In another embodiment, the beamforming and the Doppler effect and/or correlation or machine learning processing may be used together to isolate the RF Activated Range radio frequency signals responded from the specific location in the specific blood vessel to provide the high-quality signal corresponding to the blood glucose levels in the blood in the specific blood vessel. In one exemplary embodiment, Activated Range radio frequency signals of a higher frequency range of 122-126 gigahertz (GHz) in a shallower penetration depth are used to monitor blood glucose levels. It can be noted that the shallower penetration depth reduces undesirable reflections, such as reflections from bone and dense tissue such as tendons, ligaments, and muscle, which may reduce the signal processing burden and improve the quality of the desired signal that is generated from the location of the blood vessel. It can also be noted that bones are dielectric and semi-conductive. In addition, bones are anisotropic, so not only are bones conductive, but they also conduct differently depending on the direction of the flow of current through the bone. Alternatively, the bones are also piezoelectric materials. Therefore, some Activated Range radio frequency signals of higher frequency range of 122-126 GHz in the shallower penetration depth are required to monitor the blood glucose levels.

Further, the device 108 may comprise one or more transmission (TX) antennas 110, one or more receiving (RX) antennas 130, an analog to digital converter (ADC) 112, a memory 114, a processor 118, a communication module 120 and a battery 122. In one embodiment, the device 108 may be a wearable and portable device such as, but not limited to, a cell phone, a smartwatch, a tracker, a wearable monitor, a wristband, and a personal blood monitoring device. The one or more TX antennas 110 and the one or more RX antennas 130 may be fabricated on a substrate (not shown) within the device 108 in a suitable configuration. In one exemplary embodiment, at least two TX antennas 110 and at least four RX antennas 130 are fabricated on the substrate. The one or more TX antennas 110 and the one or more RX antennas 130 may correspond to a circuitry arrangement (not shown) on the substrate. Examples of specific antenna arrangements are disclosed in U.S. 2022/0192494 the entire contents of which are incorporated herein by reference.

Further, the ADC 112, the memory 114, the processor 118, the communication module 120, and the battery 122 may be fabricated on the substrate. Further, the communication module 120 may be configured to facilitate communication between the device 108 and the device network. Further, embodiments may include a plurality of TX antennas 110 and a plurality of RX antennas 130. The one or more TX antennas 110 and the one or more RX antennas 130 may be integrated into the circuitry arrangement. The one or more TX antennas 110 may be configured to transmit the RF Activated Range radio frequency signals at a pre-defined frequency. In one embodiment, the pre-defined frequency may correspond to a range suitable for the human body. For example, the one or more TX antennas 110 transmit some RF Activated Range radio frequency signals at a range of 120-126 GHz. Successively, one or more RX antennas 130 may be configured to receive the responded portion of the RF Activated Range radio frequency signals. In one embodiment, the RF Activated Range radio frequency signals may be transmitted beneath the user's skin, and electromagnetic energy may be responded from many parts such as fibrous tissue, muscle, tendons, bones, and the skin. It can be noted that effective monitoring of the blood glucose level is facilitated by an electrical response of blood molecules, such as pancreatic endocrine hormones, against the transmitted RF Activated Range radio frequency signals. It will be apparent to a skilled person that the pancreatic endocrine hormones such as insulin and glucagon are responsible for maintaining sugar or glucose level. Further, the electromagnetic energy responded from the blood molecules may be received by the one or more RX antennas 130.

Further, embodiments may include an ADC Converter 112 which may be coupled to the one or more RX antennas 130. The one or more RX antennas 130 may be configured to receive the responded RF Activated Range radio frequency signals. The ADC 112 may be configured to convert the received RF Activated Range radio frequency signals from an analog signal into a digital processor readable format. Further, embodiments may include a memory 114 that may be configured to store the transmitted RF Activated Range radio frequency signals by the one or more TX antennas 110 and store the responded portion of the transmitted RF Activated Range radio frequency signals from the one or more RX antennas 130. Further, the memory 114 may also store the converted digital processor readable format by the ADC 112. In one embodiment, the memory 114 may include suitable logic, circuitry, and/or interfaces that may be configured to store a machine code and/or a computer program with at least one code section executable by the processor 118. Examples of implementation of the memory 114 may include, but are not limited to, Random Access Memory (RAM), Read Only Memory (ROM), Hard Disk Drive (HDD), and/or a Secure Digital (SD) card. Further, embodiments may include a standard waveform database 116 that is configured to store the polling data received from the device 108.

The database may be created from an offline process in which the waveforms received from the RX antenna 130 are received and stored along with the glucose ground truth data. The ground truth data may be determined by a secondary device that identifies the glucose levels or number at or near the time a waveform was transmitted and received. Machine learning processes may be performed to identify specific glucose waveforms that can be related to glucose levels. The database is used in real time to compare received waveforms from the RX antenna 130 to waveforms in the standard waveform database 116 to identify the glucose number for the received waveform from the RX antenna 130. In one embodiment, the standard waveform database 116 may be configured to store the RF signal received from the one or more RX antennas 130 of the device 108. The standard waveform database 116 may store the transmitted signal waveforms for the TX antennas 110 and the received signal waveforms for the RX antenna 130. The database may include the glucose readings with the corresponding signal waveforms, received waveforms and the TX antenna and RX antenna that were used. Examples of implementation of the memory 114 may include, but are not limited to, Cloud storage, Cloud server, Random Access Memory (RAM), Read Only Memory (ROM), and/or a Secure Digital (SD) card.

Further, embodiments may include a processor 118 which may facilitate the operation of the device 108 with the device network to perform functions according to the instructions stored in the memory 114. In one embodiment, the processor 118 may include suitable logic, circuitry, interfaces, and/or code that may be configured to execute a set of instructions stored in the memory 114. The processor 118 may be configured to run the instructions obtained by the device base module 124 to perform polling. The processor 118 may be further configured to collect real-time signals from the one or more TX antennas 110 and the one or more RX antennas 130 and may store the real-time signals in the memory 114. In one embodiment, the real-time signals may be assigned as initial and updated radio frequency (RF) signals. Examples of the processor 118 may be an X86-based processor, a Reduced Instruction Set Computing (RISC) processor, an Application-Specific Integrated Circuit (ASIC) processor, a Complex Instruction Set Computing (CISC) processor, and/or other processors. The processor 118 may be a multicore microcontroller specifically designed to carry multiple operations based upon pre-defined algorithm patterns to achieve the desired result. Further, the processor 118 may take inputs from the device 108 and retain control by sending signals to different parts of the device 108. The processor 118 may access a Random Access Memory (RAM) that is used to store data and other results created when the processor 118 is at work. It can be noted that the data is stored temporarily for further processing, such as correlation, correction, and adjustment. Moreover, the processor 118 carries out special tasks as programs that are pre-stored in the Read Only Memory (ROM). It can be noted that the special tasks carried out by the processor 118 indicate and apply certain actions which trigger specific responses.

Further, the communication module 120 of the device 108 may communicate with the device network (not shown) via a cloud network (not shown). Examples of the communication module 120 may include, but are not limited to, the Internet, a cloud network, a Wireless Fidelity (Wi-Fi) network, a Wireless Local Area Network (WLAN), a Local Area Network (LAN), a telephone line (POTS), Long Term Evolution (LTE), and/or a Metropolitan Area Network (MAN). In one embodiment, various devices may be configured to have a communication module integrated over circuitry arrangement to connect with the device network via various wired and wireless communication protocols, such as the cloud network. Examples of such wired and wireless communication protocols may include, but are not limited to, Transmission Control Protocol and Internet Protocol (TCP/IP), User Datagram Protocol (UDP), Hypertext Transfer Protocol (HTTP), File Transfer Protocol (FTP), Zigbee, EDGE, infrared (IR), IEEE® 802.11, 802.16, cellular communication protocols, and/or Bluetooth® (BT) communication protocols.

Further, embodiments may include a battery 122 to power hardware modules of the device 108. The device 108 may be configured with a charging port to recharge the battery 122. It can be noted that the charging of the battery 122 may be achieved by wired or wireless means. In one embodiment, the battery 122 may include different models of a lithium-ion battery, such as CR1216, CR2016, CR2032, CR2025, CR2430, CR1220, CR1620, CR1616.

Further, embodiments may include the device base module 124 fabricated within the memory 114. The device base module 124 may be configured to store instructions for executing the computer program from the converted digital processor readable format of the ADC 112. The device base module 124 is configured to facilitate the operation of the processor 118, the memory 114, the one or more TX antennas 110, the one or more RX antennas 130, and the communication module 120. Further, the device base module 124 may be configured to create polling of the RF Activated Range radio frequency signals. It can be noted that the device base module 124 is configured to generate extracted signals from the RF Activated Range radio frequency signals received from the one or more RX antenna 130. Further, embodiments may include a device base module 124 which begins by starting the device 108. For example, the device base module 124 may receive the signal waveform that the TX Antennas 110 is to transmit from the processor 118. In some embodiments, the signal waveform the TX Antennas 110 is to transmit may be stored in memory. The device base module 124 sends the RF transmit signal to the TX antenna 110. The device base module 124 stores the RF transmit signal to memory 114. The device base module 124 receives the responded RF signal from the RX antenna 130. The device base module 124 converts the responded signal to digital using the ADC converter 112. The device base module 124 stores the RX converted signal data in memory 114 and the process returns to sending the RF transmit signal to the TX Antennas 110. In some embodiments, the device base module 124 may utilize the communications module 120 to communicate with a network that stores the standard waveform database 116. Some or all of the processes may be performed on a remote network (not shown). This configuration would allow the device 108 to have less processing power and memory requirements.

Further, embodiments may include a matching module 126 which begins by querying the memory 114 for a new data entry. The matching module 126 extracts the new data entry from memory 114. The matching module 126 extracts the data from the standard waveform database 116. The matching module 126 performs convolution matching. The matching module 126 stores the convolution matches in memory 114. The matching module 126 initiates the labeling module 128. Further, embodiments may include a labeling module 128 which begins by being initiated by the matching module 126. The labeling module 128 extracts the best match from memory 114. The labeling module 128 extracts the label from the best match from memory 114. The labeling module 128 stores the data in the standard waveform database 116 and then the labeling module 128 ends.

FIG. 2 displays an example operation of the device base module 124. The process executed by the device base module 124 begins with the device base module 124 starting, at step 200. For example, the device base module 124 may receive the signal waveform that the TX antenna 110 is to transmit from the processor 118. In some embodiments, the signal waveform the TX antenna 110 is to transmit may be stored in memory. The device base module 124 sends, at step 202, the RF transmit signal to the TX Antenna 110. For example, the one or more TX antennas 110 may be configured to transmit the RF Activated Range radio frequency signals at a pre-defined frequency. In one embodiment, the pre-defined frequency may correspond to a range suitable for the human body. For example, one or more TX antennas 110 transmit a specific frequency range in the Activated Range radio frequency signals at a range of 120-126 GHz. The device base module 124 stores, at step 204, the RF transmit signal to memory 114. For example, the device base module 124 stores the transmitted signal to memory 114, such as specific frequencies in the Activated Range signals at a range of 120-126 GHz. The device base module 124 receives, at step 206, the RF signal from the RX antenna 130.

For example, the one or more RX antennas 130 may be configured to receive the responded portion of the RF Activated Range radio frequency signals. In one embodiment, the RF Activated Range signals may be transmitted beneath the user's skin, and electromagnetic energy may be responded from many parts such as fibrous tissue, muscle, tendons, bones, and the skin. It can be noted that effective monitoring of the blood glucose level is facilitated by an electrical response of blood molecules, such as pancreatic endocrine hormones, against the transmitted RF Activated Range radio frequency signals. It will be apparent to a skilled person that the pancreatic endocrine hormones such as insulin and glucagon are responsible for maintaining sugar or glucose level. Further, the electromagnetic energy responded from the blood molecules may be received by the one or more RX antennas 130. The device base module 124 converts, at step 208, to digital using the ADC converter 112. For example, the ADC 112 may be configured to convert the RF Activated Range radio frequency signals from an analog signal into a digital processor readable format. The device base module 124 stores, at step 210, the RX converted signal data in memory 114 and the process returns to sending the RF transmit signal to the TX Antenna 110.

Further, an initial RF transmit signal TX is sent and then there may be a measurement of the receive antenna RX signals, so these two signals around the same time window are associated. Generally, a series of RF Transmit TX signals are sent, one right after another with the associated RX receive antenna signals are stored. The series of RF Transmit TX signals TX1, Tx2, TXn are different signals (frequencies and amplitudes). There are many possibilities of use cases as to how many and at what variabilities RF Transmit TX signals are used.

Prior to the real time use of the system, a systematic test is done to build a set of RF transmit signals that have associated receive RX antenna signals that can be analyzed, in which, the analysis can be correlated to subjects that at the same time are taking ground truth blood samples for blood glucose. With a range of subjects with a range of blood glucose levels this ground truth data is trained against the set of RF transmit signals that have associated receive RX antenna signals so that the data saved in the device network memory 128 is robust enough to be used as ground truth RX antenna signals (with their associated glucose levels) to correlate to newly obtained real time RX antenna signals.

The device base module 124 may utilize a motion module 132 that includes at least one sensor from the group of an accelerometer, a gyroscope, an inertial movement sensor, or other similar sensor. The motion module 132 may have its own processor or utilize the processor 118 to calculate the user's movement. Motion from the user will change the blood volume in a given portion of their body and the blood flow rate in their circulatory system. This may cause noise, artifacts, or other errors in the real-time signals received by the RX antennas 130. The motion module 132 may compare the calculated motion to a motion threshold stored in memory 114. For example, the motion threshold could be movement of more than two centimeters in one second. The motion threshold could be near zero to ensure the user is stationary when measuring to ensure the least noise in the RF signal data. When calculated motion levels exceed the motion threshold, the motion module 132 may flag the RF signals collected at the time stamp corresponding to the motion as potentially inaccurate. In some embodiments, the motion module 132 may compare RF signal data to motion data over time to improve the accuracy of the motion threshold. The motion module 132 may alert the user, such as with an audible beep or warning or a text message or alert to a connected mobile device. The alert would signal the user that they are moving too much to get an accurate measurement. The motion module 132 may update the standard waveform database 116 with the calculated motion of the user that corresponds with the received RF signal data. In this manner, the motion module 132 may be simplified to just collect motion data and allow the device base module 124 to determine if the amount of motion calculated exceeds a threshold that would indicate the received RF signal data is too noisy to be relied upon for a blood glucose measurement.

The device base module 124 may utilize a body temperature module 134 that includes at least one sensor from the group of a thermometer, a platinum resistance thermometer (PRT), a thermistor, a thermocouple, or another temperature sensor. The body temperature module 134 may have its own processor or utilize the processor 118 to calculate the temperature of the user or the user's environment. The user's body temperature, the environmental temperature, and the difference between the two will change the blood volume in a given part of their body and the blood flow rate in their circulatory system. Variations in temperature from the normal body temperature or room temperature may cause noise, artifacts, or other errors in the real-time signals received by the RX antennas 130. The body temperature module 134 may compare the measured temperature to a threshold temperature stored in memory 114. For example, the environmental temperature threshold may be set at zero degrees Celsius because low temperatures can cause a temporary narrowing of blood vessels which may increase the user's blood pressure. When the measured temperature exceeds the threshold, the body temperature module 134 may flag the RF signals collected at the time stamp corresponding to the temperature as potentially being inaccurate. In some embodiments, the body temperature module 134 may compare RF signal data to temperature data over time to improve the accuracy of the temperature threshold. The body temperature module 134 may alert the user, such as with an audible beep or warning or a text message or alert to a connected mobile device. The alert would signal to the user that their body temperature or the environmental temperature is not conducive to getting an accurate measurement. The body temperature module 134 updates the standard waveform database 116 with the measured user or environmental temperature that corresponds with the received RF signal data. In this manner, the body temperature module 134 may be simplified to just collect temperature data and allow the device base module 124 to determine if the temperature measure exceeds a threshold that would indicate the received RF signal data is too noisy to be relied upon for a blood glucose measurement.

The device base module 124 may utilize a body position module 136 that includes at least one sensor from the group of an accelerometer, a gyroscope, an inertial movement sensor, or another similar sensor. The body position module 136 may have its own processor or utilize the processor 118 to estimate the user's position. The user's body position may change the blood volume in a given part of their body and the blood flow rate in their circulatory system. This may cause noise, artifacts, or other errors in the real-time signals received by the RX antennas 130. The body position module 136 may compare the estimated position to a body position threshold stored in memory 114. For example, the monitoring device 102 may be on the user's wrist, and the body position threshold may be based on the relative position of the user's hand to their heart. When a user's hand is lower than their heart, their blood pressure will increase, with this effect being more pronounced the longer the position is maintained. Conversely, the higher a user holds their arm above their heart, the lower the blood pressure in their hand. The body position threshold may include some minimum amount of time the estimated body position occurs. When the estimated position exceeds the threshold, the body position module 136 may flag the RF signals collected at the time stamp corresponding to the body position as potentially being inaccurate. In some embodiments, the body position module 136 may compare RF signal data to motion data over time to improve the accuracy of the body position threshold. The body position data may also be used to estimate variations in parameters such as blood pressure that corresponds to the body position data to improve the accuracy of the measurements taken when the user is in that position. The body position module 136 may alert the user, such as with an audible beep or warning or a text message or alert to a connected mobile device. The alert would signal to the user that their body position is not conducive to getting an accurate measurement. The body position module 136 may update the standard waveform database 116 with the estimated body position data that corresponds with the received RF signal data. In this manner, the body temperature module 134 may be simplified to just collect temperature data and allow the device base module 124 to determine if the body position exceeded a threshold that would indicate the received RF signal data is too noisy to be relied upon for a blood glucose measurement.

The device base module 124 may utilize an ECG module 138 that includes at least one electrocardiogram sensor. The ECG module 138 may have its own processor or utilize the processor 118 to record the electrical signals that correspond with the user's heartbeat. The user's heartbeat will impact blood flow. Measuring the ECG data may allow the received RF data to be associated with peak and minimum cardiac output so as to create a pulse waveform allowing for the estimation of blood volume at a given point in the wave of ECG data. Variations in blood volume may cause noise, artifacts, or other errors in the real-time signals received by the RX antennas 130. The ECG module 138 may compare the measured cardiac data to a threshold stored in memory 114. For example, the threshold may be a pulse above 160 bpm, as the increased blood flow volume may cause too much noise in the received RF signal data to accurately measure the blood glucose. When the ECG data exceeds the threshold, the ECG module 138 may flag the RF signals collected at the time stamp corresponding to the ECG data as potentially being inaccurate. In some embodiments, the ECG module 138 may compare RF signal data to ECG data over time to improve the accuracy of the ECG data threshold or to improve the measurement of glucose at a given point in the cycle between peak and minimum cardiac output. The ECG module 138 may alert the user, such as with an audible beep or warning or a text message or alert to a connected mobile device. The alert would signal to the user that their heart rate is not conducive to getting an accurate measurement or requires additional medical intervention. The ECG module 138 may update the standard waveform database 116 with the measured ECG data that corresponds with the received RF signal data. In this manner, the ECG module 138 may be simplified to just collect ECG data and allow the device base module 124 to determine if the ECG data exceeded a threshold that would indicate the received RF signal data is too noisy to be relied upon for a blood glucose measurement.

The device base module 124 may utilize a circadian rhythm module 140 that includes at least one sensor measuring actigraphy, wrist temperature, light exposure, and heart rate. The circadian rhythm module 140 may have its own processor or utilize the processor 118 to calculate the user's circadian health. Blood pressure follows a circadian rhythm in that it increases upon waking in the morning and decreases during sleeping at night. People with poor circadian health will often have higher blood pressure. These variations in blood pressure can cause noise, artifacts, or other errors or inaccuracies in the real-time signals received by the RX antennas 130. The circadian rhythm module 140 may compare the circadian data to a threshold stored in memory 114. For example, the threshold may be less than 6 hours of sleep in the last 24 hours. When the observed circadian health data exceeds the threshold, the circadian rhythm module 140 may flag the RF signals collected at the time stamp corresponding to circadian health as potentially being inaccurate or needing an adjustment to account for the expected increase in the user's blood pressure. In some embodiments, the circadian rhythm module 140 may compare RF signal data to sleep data over time to improve the accuracy of the circadian rhythm thresholds. The circadian rhythm module 140 may alert the user, such as with an audible beep or warning, or a text message or alert to a connected mobile device. The alert would signal to the user that their recent sleep patterns are not conducive to getting an accurate measurement. The circadian rhythm module 140 may update the standard waveform database 116 with the measured circadian data that corresponds with the received RF signal data. In this manner, the circadian rhythm module 140 may be simplified to just collect circadian rhythm data and allow the device base module 124 to determine if the measure exceeded a threshold that would indicate the received RF signal data is too noisy to be relied upon for a blood glucose measurement, or if an alternative transfer function should be used to compensate for the detected circadian health.

The device base module 124 may include a received noise module 142 that includes at least one sensor measuring background signals such as RF signals, Wi-Fi, and other electromagnetic signals that could interfere with the signals received by the RX antennas 130. The received noise module 142 may have its own processor or utilize the processor 118 to calculate the level of background noise being received. Background noise may interfere with or cause noise, artifacts, or other errors or inaccuracies in the real-time signals received by the RX antennas 130. The received noise module 142 may compare the level and type of background noise to a threshold stored in memory 114. The threshold may be in terms of field strength (volts per meter and ampere per meter) or power density (watts per square meter). For example, the threshold may be RF radiation greater than 300 μW/m2. When the background noise data exceeds the threshold, the received noise module 142 may flag the RF signals collected at the time stamp corresponding to background noise levels as potentially being inaccurate. In some embodiments, the received noise module 142 may compare RF signal data to background noise over time to improve the accuracy of the noise thresholds. The received radiation module may alert the user, such as with an audible beep or warning, a text message, or an alert to a connected mobile device. The alert would signal to the user that the current level of background noise is not conducive to getting an accurate measurement. The received noise module 142 may update the standard waveform database 116 with the background noise data that corresponds with the received RF signal data. In this manner, the received noise module 142 may be simplified to just collect background noise data and allow the device base module 124 to determine if the measure exceeded a threshold that would indicate the received RF signal data is too noisy to be relied upon for a blood glucose measurement, or if an alternative transfer function should be used to compensate for the noise.

In order to start the process, the Transmit TX signals TX1, TX2, TXn are started from an initial TX1 signal and the receive antenna signal associated with this TX1 signal is obtained. The initial TX1 signal is then updated to say TX2 and the process repeats until all transmit TX signals are sent.

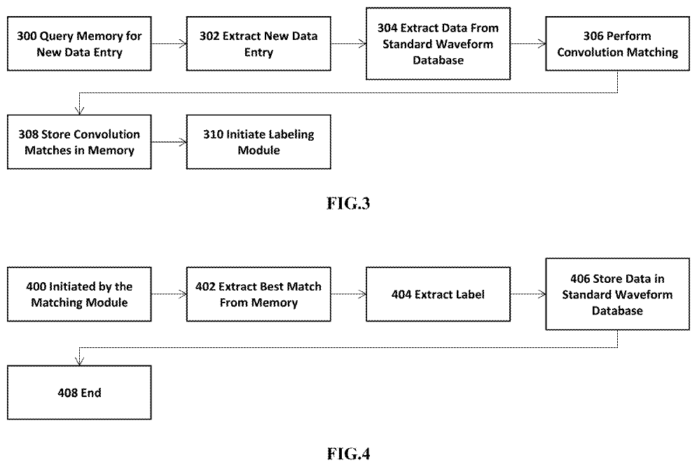

FIG. 3 illustrates the matching module 126. The process executed by the matching module 126 begins with the matching module 126 querying, at step 300, the memory 114 for a new data entry. For example, the matching module 126 queries the memory 114 for the new data entry, such as the RF transmit signal that was used by the TX Antenna 110 and the received waveform from the RX antenna 130 that was converted to a digital processor readable format. The matching module 126 extracts, at step 302, the new data entry from memory 114. For example, the matching module 126 extracts the RF transmit signal that was used by the TX Antenna 110 and the received waveform from the RX antenna 130 that was converted to a digital processor readable format from the memory 114. The matching module 126 extracts, at step 304, the data from the standard waveform database 116. For example, the matching module 126 extracts the data in the standard waveform database 116, such as the waveforms received from the RX antenna 130 that are received and stored along with the glucose ground truth data. The ground truth data may be determined by a secondary device that identifies the glucose levels or number at the time a waveform was transmitted and received. Machine learning processes may be performed to identify specific glucose waveforms, which complex responded signals from stepped frequencies transmit signals, that can be related to glucose levels. The database is used in real time to compare received waveforms from the RX antenna 130 to the standard waveform database 116 to identify the glucose number for the received waveform from the RX antenna 130. The matching module 126 performs, at step 306, convolution matching. For example, the matching module 126 performs convolution matching on the extracted RF transmit signal that was used by the TX Antenna 110 and the received waveform from the RX antenna 130 that was converted to a digital processor readable format and the data extracted from the standard waveform database 116. For example, convolution is a mathematical operation on two functions, f and g, that produces a third function, f*g, that expresses how the shape of one is modified by the other. The term convolution refers to both the result function and to the process of computing it. It is defined as the integral of the product of the two functions after one is responded about the y-axis and shifted. The choice of which function is responded and shifted before the integral does not change the integral result. The integral is evaluated for all values of shift, producing the convolution function. The waveforms stored in memory 114 are matched with each of the individual data entries stored in the standard waveform database 116. First, the input signal can be decomposed into a set of impulses, each of which can be viewed as a scaled and shifted delta function. Second, the output resulting from each impulse is a scaled and shifted version of the impulse response. Third, the overall output signal can be found by adding these scaled and shifted impulse responses. In other words, if a system's impulse response is known, then we can calculate what the output will be for any possible input signal. In some embodiments, the matching module 126 may also perform cross-correlation and autocorrelation and store the all the results in memory 114. The matching module 126 stores, at step 308, the convolution matches in memory 114. For example, the matching module 126 stores all the results from convolution in memory 114, such as all of the resulting third functions from the waveform stored in memory 114 and for each data entry stored in the standard waveform database 116. The matching module 126 initiates, at step 310, the labeling module 128.

In another embodiment (not shown), instead of convolution matching to get an extracted signal, filtering is used on at least one of the pulse wave signal and a mathematical model generated in response to the extracted pulse wave signal with a lowpass filter to generate a filtered extracted signal. The mathematical model may be a complex algebraic expression of the signal amplitude over time that may demonstrate, say 5 peaks of signal separated by various time windows, where each peak is within some range and each peak has an upward slope and downward slope. So if it is found, from a ground truth blood test, that say a glucose reading of 75 correlates to a received RX signal of 6 peaks, each with a unique time window and slope, a mathematical model may be created and saved in the standard waveform database 116. Not shown, there could be numerous mathematical models saved in the standard waveform database, where each mathematical model would represent a glucose label. So each new received RX signal is analyzed against each mathematical model in the standard waveform database 116. If the new received RX signal analyzed against at least one matching mathematical waveform, within a threshold matching (done with correlation algorithms), then that best correlation to the closest matching to the standard waveform database 116 would be considered the closest match, and therefore the closest match glucose labelling (e.g. 75 glucose reading) would be associated with the new received RX signal.

FIG. 4 illustrates the labeling module 128. The process executed by the labeling module 128 begins with the labeling module 128 being initiated, at step 400, by the matching module 126. In some embodiments, the labeling module 128 may be initiated once the convolution process is completed by the matching module 126. The labeling module 128 extracts, at step 402, the best match from memory 114. For example, the best match may be determined by the amount the created third function from convolution is similar to the original function, or the original waveform that was stored in memory 114. The best match may be the data entry from the standard waveform database 116 which includes the waveforms received from the TX antenna 110 and the RX antenna 130 that are received and stored along with the glucose ground truth data. The ground truth data may be determined by a secondary device that identifies the glucose levels or number at the time a waveform was transmitted and received. The labeling module 128 extracts, at step 404, the label from the best match from memory 114. For example, the labeling module 128 may extract the label, such as the glucose level or number, from the best match from memory 114. The labeling module 128 stores, at step 406, the data in the standard waveform database 116. For example, the labeling module 128 stores the original waveform received from the RX antenna 130 that was converted using the ADC converter 112 and the glucose level or number in the standard waveform database 116. The labeling module 128 ends, at step 408. For example, once the data entry stored in memory has been labeled and stored in the standard waveform database 116 the process ends.

The functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

1. A health monitoring system, comprising:
a monitoring device that includes one or more transmit antennas configured to transmit radio-frequency (RF) glucose detection signals into a user and one or more receive antennas configured to detect RF glucose signals that result from the RF glucose detection signals transmitted into the user;
an analog-to-digital converter connected to the one or more receive antennas and receiving the RF glucose signals detected by the one or more receive antennas;
memory that stores a standard waveform database with a plurality of standard waveforms;
a matching module stored in the memory and that is configured to perform convolution matching;
a labeling module stored in the memory and that access labels stored in the memory.

2. The health monitoring method of claim 1, comprising a plurality of the receive antennas.

3. A health monitoring method, comprising:
generating a waveform database of glucose waveforms and associated health parameter labels;
transmitting radio-frequency (RF) glucose detection signals into a user from one or more transmit antennas and detecting, using one or more receive antennas, RF glucose signals that result from the RF glucose detection signals transmitted into the user;
converting the detected RF glucose signals from analog signals to digital signals using an analog-to-digital converter connected to the one or more receive antennas;
extracting one of the glucose waveforms from the waveform database and performing convolution matching on the extracted glucose waveform and one of the digital signals;
storing a convolution match in memory;
labeling data corresponding to the convolution match with a corresponding blood glucose level.

4. A method of generating training data for use in monitoring a health parameter of a person, the method comprising:
generating a waveform database of glucose waveforms and health parameter labels;
receiving a pulse wave signal that is generated from radio frequency scanning data that corresponds to radio waves that have responded from below the skin surface of a person, wherein the radio frequency scanning data is collected through a two-dimensional array of receive antennas over a range of radio frequencies;
storing the pulse wave signal in the waveform database;
extracting the pulse wave signal from the waveform database and performing convolution matching to generate an extracted signal; and
labeling data corresponding to the extracted signal with a corresponding blood glucose level to generate training data.
